(19) United States
(12) Patent Application Publication
(10) Pub. No.: US 2016/0128613 A1
(43) Pub. Date: May 12, 2016

(54) BODY INFORMATION SENSING DEVICE

(71) Applicant: PEGATRON CORPORATION, Taipei City (TW)

(72) Inventors: HSIN-TSUNG TUNG, Taipei City (TW); YI-HSIANG HUANG, Taipei City (TW)

(21) Appl. No.: 14/932,535

(22) Filed: Nov. 4, 2015

(30) Foreign Application Priority Data
Nov. 7, 2014 (TW) 103138802

(51) Int. Cl. A61B 5/1455; A61B 5/024; A61B 5/00; A61B 5/01

(52) U.S. Cl. CPC A61B 5/14551; A61B 5/01; A61B 5/02416; A61B 5/7207; A61B 5/7455; A61B 5/6887

(57) ABSTRACT

A body information sensing device for obtaining body information of a user. The body information sensing device includes a housing, a sensing module and a processing module. The housing is detachably disposed at the outer surface of a daily necessity. The sensing module is disposed at the outer surface of the housing, wherein the sensing module is for sensing the body information of the user. The processing module is disposed at the housing, and the sensing module is electrically connected with the processing module.

### BACKGROUND

1. Technical Field

The disclosure relates to a sensing device and, in particular, to a detachable device for sensing body information.

2. Related Art

Nowadays people are increasingly concerned about their health and the development of science and technology makes it possible to obtain more accurate health information of a body such as heartbeat, body temperature or blood oxygen by instrument measurements. However, incidence of a disease, such as a stroke or a myocardial, often comes in a sudden. While the health information above can be used to monitor their body conditions, it is necessary to prepare household measuring instrument.

However, modern household measuring instruments are often stand-alone devices. A user needs to especially go to the disposition position of an instrument and spend extra time to perform a measurement. If the user does not have the habit to measure timely or does not have time to measure, he or she will not be able to know the body condition of his or her own.

### SUMMARY

The objective of the invention is to provide a body information sensing device that can be used by a user naturally.

To achieve the objective above, the invention provides a body information sensing device for obtaining body information of a user. The body information sensing device includes a housing, a sensing module and a processing module. The housing is detachably disposed at the outer surface of a daily necessity. The sensing module is disposed at the outer surface of the housing, wherein the sensing module is for sensing the body information of the user. The processing module is disposed at the housing, and the sensing module is electrically connected with the processing module.

In an embodiment of the invention, the housing is made of elastically-retractable material.

In an embodiment of the invention, the sensing module includes a body temperature sensing unit, a blood oxygen sensing unit or a heartbeat sensing unit.

In an embodiment of the invention, the heartbeat sensing unit is correspondingly disposed at the contact position of the thumb of the user, and the blood oxygen sensing unit correspondingly disposed at the contact position of the index finger of the user.

In an embodiment of the invention, the body temperature sensing unit is correspondingly disposed at a contact position of the palm of the user.

In an embodiment of the invention, the body information sensing device further includes a light-emitting module. The light-emitting module is disposed at the housing, electrically connected with the processing module, and includes a shield-sensing unit.

In an embodiment of the invention, the body information sensing device further includes a vibration unit. The vibration unit is disposed at the housing and electrically connected with the processing module.

In an embodiment of the invention, the body information sensing device further includes an acceleration sensing unit. The processing module controls the vibration unit via the acceleration sensing unit.

In an embodiment of the invention, the body information sensing device further includes a message transceiver module. The processing module controls the message transceiver module to transmit the information sensed by the sensing module to a mobile electronic device.

In an embodiment of the invention, the body information sensing device further includes a projection module. The processing module controls the projection module, and the projection module projects the information sensed by the sensing module.

In an embodiment of the invention, the body information sensing device further includes a light-emitting display unit. The light-emitting display unit is disposed at the outer surface of the housing for prompting a message.

Through the above embodiments, the invention has at least the advantages below: since the housing is detachably disposed at a daily necessity and the sensing module is disposed on the housing, a user can use the body information sensing device naturally and can select the daily necessity to dispose the body information sensing device freely.

### BRIEF DESCRIPTION OF THE DRAWINGS

FIG. 1 is a schematic module diagram showing the body information sensing device according to an embodiment of the invention.

FIG. 2 is a schematic perspective diagram showing the body information sensing device according to an embodiment of the invention.

FIG. 3 is a schematic expanded diagram showing the body information sensing device according to an embodiment of the invention.

FIG. 4 is a schematic diagram showing the use of the body information sensing device according to an embodiment of the invention.

FIG. 5 is a schematic diagram showing use of the body information sensing device according to an embodiment of the invention.

FIG. 6 is a schematic diagram showing use of the body information sensing device according to an embodiment of the invention.

### DETAILED DESCRIPTION OF THE EMBODIMENT

The present invention will be apparent from the following detailed description, which proceeds with reference to the accompanying drawings, wherein the same references relate to the same elements.

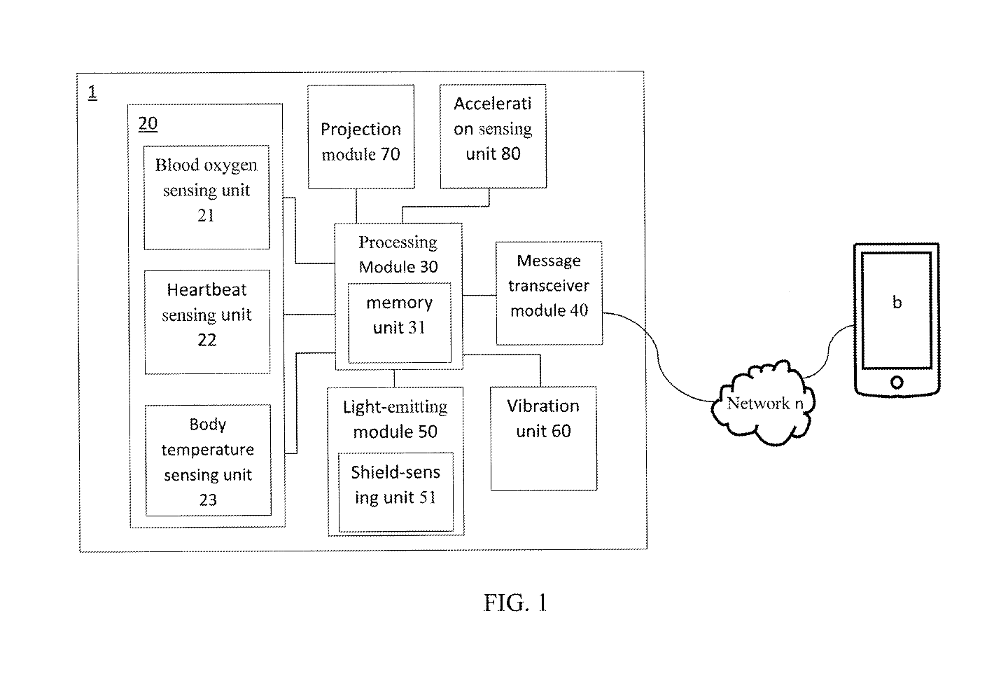

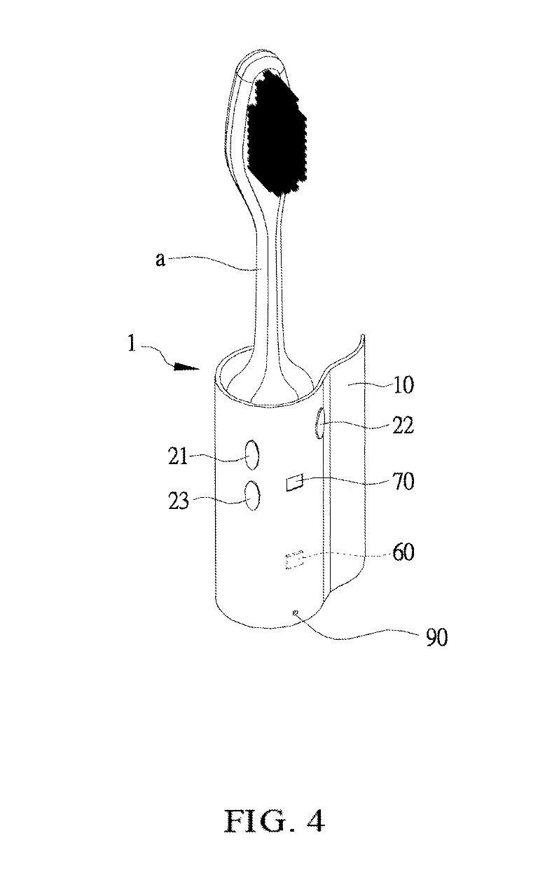

Referring to FIG. 1, the body information sensing device 1 according to an embodiment of the invention is detachably disposed at a daily necessity a (referring to FIG. 4), and can transmit messages with a mobile electronic device b.

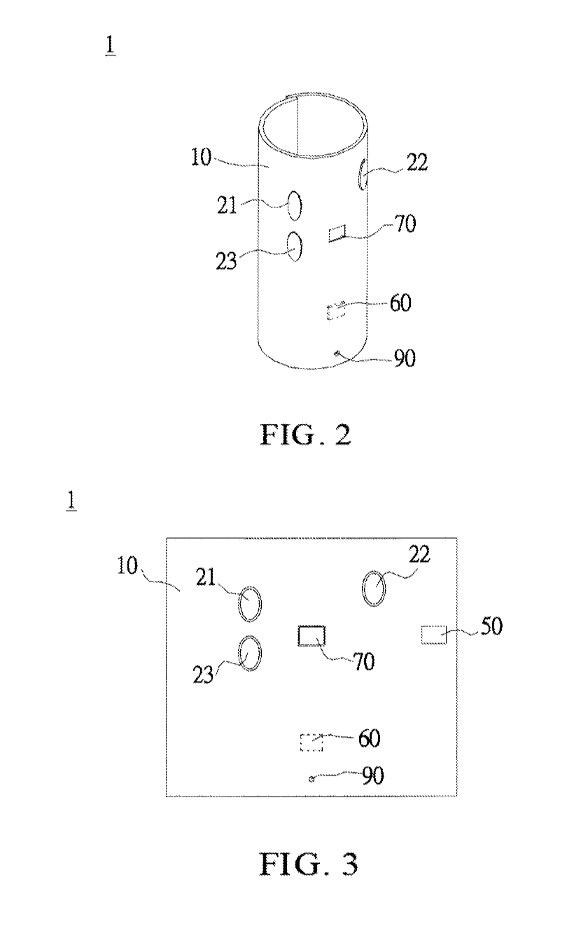

Referring to FIG. 1 and FIG. 2, the body information sensing device 1 includes a housing 10, a sensing module 20, a processing module 30, a message transceiver module 40, a light-emitting module 50, a vibration module 60, a projection module 70, an acceleration sensing unit 80 and a light-emitting display unit 90 (as shown in FIG. 2). The sensing module 20, the processing module 30, the message transceiver module 40, the light-emitting module 50, the vibration module 60, the projection module 70 and the acceleration sensing unit 80 are disposed at the housing 10 respectively, and are electrically connected with each other.

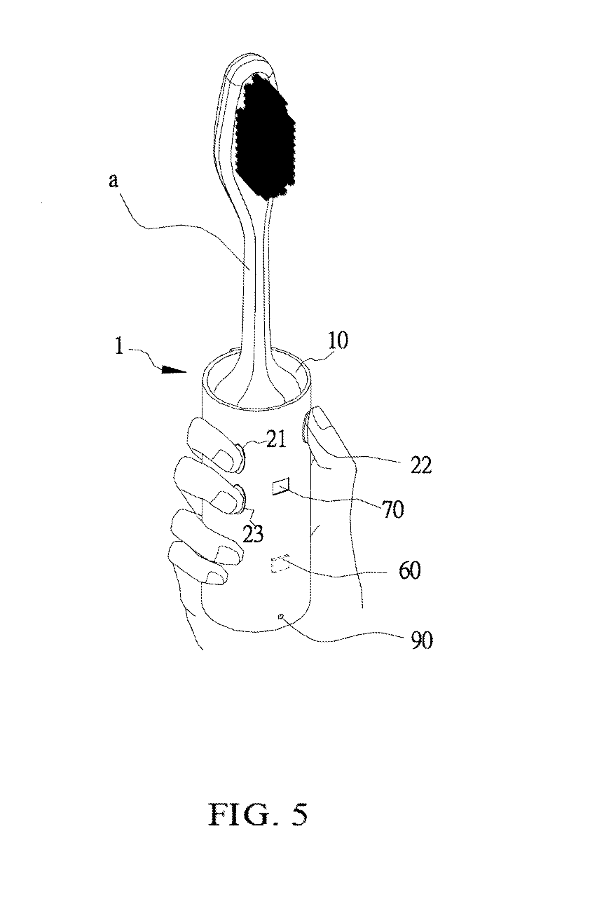

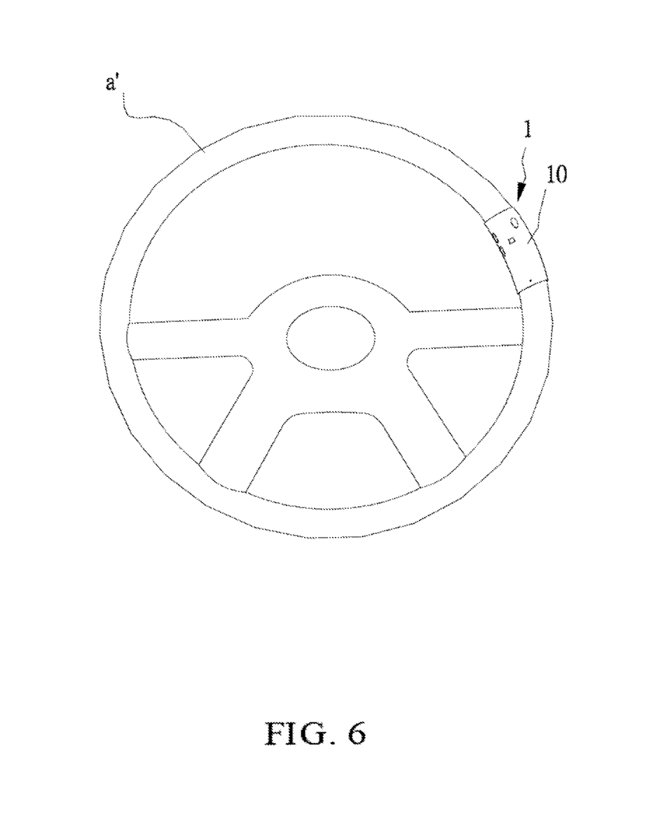

The housing 10 has a detachable fixing structure that allows the body information sensing device 1 to be detachably disposed at a daily necessity a repeatedly. The fixing structure of the housing 10 may be any structure that does not damage the structure of the daily necessity, such as a clamp structure or a sheath structure. Referring to FIG. 2 and FIG. 3, in the embodiment of the invention, the housing 10 is made of elastically-retractable material, such as a steel tape, a spring steel or an elastic metal bar, so that the housing 10 can be expanded or retracted when being bent. Such design makes the body information sensing device 1 be capable of being fixed at different kinds of bar-shaped objects conveniently. A user can select the daily necessity to fix the body information sensing device 1 without being effected by the replacement of the daily necessity a. As shown in FIG. 4 and FIG. 5, the body information sensing device 1 can be fixed to a daily necessity a such as a toothbrush. Or, it can be fixed to a daily necessity a′ such as a steering wheel as shown in FIG. 6.

As shown in FIG. 1, the sensing module 20 includes a blood oxygen sensing unit 21, a heartbeat sensing unit 22 and a body temperature sensing unit 23. In an embodiment of the invention, the sensing module 20 is used for getting close to or contacting with the body of the user to sense the body information of the user. The blood oxygen sensing unit 21 estimates the blood oxygen via the absorption rate of the blood regarding a specific spectrum obtained by emitting and receiving light. The detailed operation principles of the blood oxygen sensing unit 21, the heartbeat sensing unit 22 and the body temperature sensing unit 21 are known in the prior art, and are omitted here for concise purpose.

The processing module 30 includes a memory unit 31. The light-emitting module 50 includes a shield-sensing unit 51. The shield-sensing unit 51 can sensing whether the light-emitting module 50 is shielded, and further determines that the user covers the light-emitting module 50 by gripping when the light-emitting module 50 is shielded.

The positions of the electronic components and the housing 10 are explained hereinbelow. Referring to FIG. 4 and FIG. 5, the body information sensing device 1 is detachably disposed at the outer surface of the daily necessity a. Therefore, when the user grips the daily necessity a attached with the body information sensing device 1, the positions of his or her fingers can be substantially the same to those when gripping the daily necessity a originally without the information sensing device 1. The blood oxygen sensing unit 21 is disposed at a position corresponding to the contact position of the index finger of the user, so that the user can contact it with the index finger. The heartbeat sensing unit 22 is disposed at a position corresponding to the contact position of the thumb of the user, so that the user can contact it with the thumb. The body temperature sensing unit 23 is disposed at a position corresponding to the contact position of the middle finger of the user, and preferably at a position extended to the palm of the user to facilitate the contact of the palm of the user. Since the palm temperature is closer to the body temperature (which can avoid the issue that the peripheral temperature is lower), the temperature measured by the body temperature sensing unit 23 may be more accurate.

The processing module 30, the message transceiver module 40, the vibration unit 60 and the light-emitting display unit 90 are disposed in the housing 10. The light-emitting module 50 and the projection module 70 are disposed on the housing 10 to expose to the outer surface of the housing 10. The light-emitting module 50 can be correspondingly disposed at the portion that can be covered by the user's hand, so that the user can cover the light-emitting module 50 when holding the daily necessity a. At this moment, the shield-sensing unit 51 senses the shielding, and sends the shielded information to the processing module 30, which operations will be described later. The projection module 70 is disposed at a position that is not covered by the user's hand, so that it would not be shielded by the user's hand during the projection operation. The light-emitting display unit 90 may be used to prompt the power status, any abnormal condition of the body, or the incomplete transmission of the data from the memory unit 31 to the mobile electronic device b. The light-emitting display unit 90 may indicate different conditions by different light colors. In the embodiment of the invention, the light-emitting display unit 90 is an LED.

The operations of the body information sensing device 1 will be described hereinbelow. Referring to FIG. 1 and FIG. 4, the user can pair the body information sensing device 1 with a mobile electronic device b, and dispose the body information sensing device 1 on a daily necessity a via the structure of the housing 10 to finish the preparation prior to using the body information sensing device 1.

When the user picks up the daily necessity a, the acceleration sensing unit 80 senses the variation of the acceleration of the daily necessity a. The processing module 30 controls the message transceiver module 40 to establish the connection with the mobile electronic device b according to the sensed information of the acceleration sensing unit 80. After the connection being established successfully, the processing module 30 controls the vibration unit 70 to let the user know that the connection has been established successfully, and sends the data stored in the memory unit 31 to the mobile electronic device b. If the data transmission fails, the light-emitting display unit would light up to attract the user's attention. In FIG. 1, the message transceiver module 40 is connected with the mobile electronic device b via a network n. The network n may be wireless or a wired communication network, such as a Bluetooth, a Wi-Fi, or a USB network.

When the user grips the daily necessity a, the hand covers the light-emitting module 50. The processing module 30 controls the sensing module 20 via the sensed information of the shield-sensing unit 51 to sense the body information of the user via the blood oxygen sensing unit 21, the heartbeat sensing unit 22 and the body temperature sensing unit 23. The processing module 30 receives the body information sensed by the sensing module 20, and sends the body information to the mobile electronic device b via the message transceiver module 40. If the body information sensing device 1 is not paired with the mobile electronic device 1 in advance, the processing module 30 can store the body information in the memory unit 31.

In the embodiment, the body information sensing device 1 can also project the sensed body information to a surface such as a mirror or a wall via the projection module 70. Furthermore, if the mobile electronic device b has other body information of the user (such as the height, the weight or the age of the user), an application of the mobile electronic device b can perform calculations based on the body information to determine whether the heartbeat is normal or whether the temperature is too low, etc., and return the results back to the body information sensing device 1. The calculated results can also be projected via the projection module 70. If the mobile electronic device b analyzes to determine the body information of the user is abnormal, the user can be notified by the vibration of the vibration unit 60 or the light of the light-emitting display unit 90.

Referring to FIG. 4, the body information sensing device 1 is disposed at a daily necessity a such as a toothbrush. Moreover, since people usually bring mobile electronic devices b with them, a user can use the body information sensing device 1 naturally and have the sensed information be sent to the mobile electronic device b naturally.

To sum up, the body information sensing device at least have the following advantages: since the housing is detachably disposed at a daily necessity and the sensing module is disposed on the housing, a user can use the body information sensing device naturally and can select the daily necessity to dispose the body information sensing device freely.

Although the invention has been described with reference to specific embodiments, this description is not meant to be construed in a limiting sense. Various modifications of the disclosed embodiments, as well as alternative embodiments, will be apparent to persons skilled in the art.

It is, therefore, contemplated that the appended claims will cover all modifications that fall within the true scope of the invention.

What is claimed is:

1. A body information sensing device for obtaining body information of a user, the body information sensing device comprising:
a housing detachably disposed at the outer surface of a daily necessity;
a sensing module disposed at the outer surface of the housing, wherein the sensing module is for getting close to or contacting with the body of the user to sense the body information of the user;
a processing module disposed at the housing, the sensing module being electrically connected with the processing module; and
a message transceiver module disposed at the housing, the message transceiver module being electrically connected with the processing module,
wherein the processing module controls the message transceiver module to send the body information sensed by the sensing module to a mobile electronic device.

2. The body information sensing device according to claim 1, wherein the housing is made of elastically-retractable material.

3. The body information sensing device according to claim 2, wherein the elastically-retractable material comprises a steel tape structure, a spring steel or an elastic metal bar.

4. The body information sensing device according to claim 2, wherein the sensing module comprises a body temperature sensing unit, a blood oxygen sensing unit or a heartbeat sensing unit.

5. The body information sensing device according to claim 4, wherein the heartbeat sensing unit is for being contacted by a thumb of the user, the blood oxygen sensing unit if for being contacted by an index finger of the user.

6. The body information sensing device according to claim 5, wherein the body temperature sensing unit is for being contacted by a palm of the user.

7. The body information sensing device according to claim 6, further comprising a light-emitting module, the light-emitting module being disposed at the housing, the light-emitting module being electrically connected with the processing module, the light-emitting module including a shield-sensing unit.

8. The body information sensing device according to claim 7, further comprising a vibration unit, the vibration unit being disposed at the housing, the vibration unit being electrically connected with the processing module.

9. The body information sensing device according to claim 8, further comprising an acceleration sensing unit, the acceleration sensing unit being electrically connected with the processing module.

10. The body information sensing device according to claim 9, further comprising a projection module, the projection module being electrically connected with the processing module, the processing module controlling the projection module to project information sensed by the sensing module.

11. The body information sensing device according to claim 1, further comprising a light-emitting display unit, the light-emitting display unit being disposed at the outer surface of the housing, the light-emitting display unit being for prompting a message.
